(19) United States
(12) Patent Application Publication
(10) Pub. No.: US 2011/0166421 A1
(43) Pub. Date: Jul. 7, 2011

(54) Enhanced Otoscope Cover

(76) Inventor: Pejman Katiraei, Los Angeles, CA (US)

(21) Appl. No.: 12/651,448

(22) Filed: Jan. 1, 2010

(51) Int. Cl. A61B 1/227

(52) U.S. Cl. 600/200

(57) ABSTRACT

Some embodiments provide an attachment for adapting an examination tool to identify a foreign object within a small orifice and to safely extract the foreign object using the examination tool. In some embodiments, the attachment is an enhanced otoscope cover for adapting an otoscope to simultaneously examine the ear canal and remove accumulated cerumen from the ear canal. The attachment includes a cover to couple the attachment to the otoscope and to provide a focal view point for the attachment. The attachment includes a support that extends from the cover. An extraction tip is located at the end of the support and is used to engage and extract cerumen and other objects.

### TECHNICAL FIELD

The present invention relates to an attachment for medical examination tools. More particularly, this invention relates to an enhanced otoscope cover for performing examination and extraction.

### BACKGROUND ART

Cerumen or ear wax is naturally secreted within the ear canal to trap dirt and other foreign objects (e.g., insects, water, etc.) from entering deep into the ear canal. Cerumen also serves to inhibit bacterial growth or fungal growth within the ear canal. The natural motion of the jaw, through talking or chewing, normally causes the cerumen to be forced out of the ear canal. However, excessive accumulation of cerumen or ear wax is a problem frequently encountered during medical examinations. Excess cerumen within the ear canal may create blockages that impair one's ability to hear.

An otoscope is a medical examination tool that is found in the offices of most healthcare practitioners. Healthcare practitioners use the otoscope to visualize the ear canal and ear drum and to identify any blockages caused by excess cerumen and other foreign objects within the ear canal. The otoscope provides light and magnification to help visualize small orifices in order to better identify such blockages. However, the otoscope currently does not provide a means for removing the blockages.

Instead, curettes are the preferred tools used by healthcare practitioners to remove accumulated cerumen or other blockages. In specialized cases, healthcare practitioners use high pressure water jets to remove the accumulated cerumen or other blockages. Curettes are specialized probes that are inserted into the ear canal to extract the cerumen. At the end of a curette is a tip used to scoop the cerumen away from the ear canal. Some curettes provide a light to illuminate the inside of the ear canal in order to more easily identify and extract the cerumen. Some curettes provide a magnification lens to enlarge the viewing of the ear canal and provide for better precision in extracting the cerumen. Accordingly, curettes are specialized tools that often serve the single purpose of removing cerumen and therefore become an additional expense to the healthcare practitioner.

It is therefore an objective of the present invention to enhance the functionality of the otoscope so as to adapt the light and magnification functionalities of the otoscope for simultaneous object identification and object extraction. Accordingly, it is an objective of the present invention to reduce the time and cost associated with the procedure of removing an object from a small orifice or other openings within the human body by eliminating the need for specialized tools such as curettes.

### SUMMARY OF THE INVENTION

Some embodiments provide an attachment for adapting an examination tool to identify an object within a small orifice or other opening and to safely extract the object using the examination tool. In some embodiments, the attachment is an enhanced otoscope cover with various extraction tips for performing object extraction. In some embodiments, the attachment couples to an otoscope in order to leverage the light and magnification functionalities of the otoscope. In this configuration, the otoscope may be used to identify and precisely extract small objects from various openings or orifices using the extraction tip provided by the attachment. Specifically, the attachment may be used to examine the ear canal and remove accumulated cerumen from the ear canal. Accordingly, a healthcare practitioner is able to perform examination and extraction without additional specialized tools such as curettes. The attachment includes a cover, a support, and an extraction tip. In some embodiments, the cover is an otoscope cover also referred to as a speculum cover. The cover includes a focal tip and a grooved base. The focal tip narrows the circumference of the cover so that it may be inserted into a small orifice (e.g., ear canal) or other opening. The focal tip focuses the view of the otoscope to what can be seen through the focal tip. Light from the otoscope illuminates the view from the focal tip. The grooved base couples the attachment to the head of the otoscope.

The support extends a specified distance from the focal tip of the cover. In some embodiments, the support extends at an angle from the focal tip. In some embodiments, the support is retractable. In some such embodiments, the support slides across the side of the cover.

In some embodiments, the extraction tip is positioned to engage cerumen and other objects from behind. Once engaged, the attachment is pulled thereby creating a traction force with the extraction tip which extracts the cerumen or other objects. The extraction tip may be of various shapes including: circular, oblong, square, rectangular, hollow, solid, or some combination thereof. The tip may include bristles.

In some embodiments, the extraction tip protrudes at an angle from the end of the support. The extraction tip is angled from the support so that it is seen through the focal tip of the cover. The healthcare practitioner is thus able to precisely position the extraction tip to engage the cerumen using the magnified view provided by the otoscope. In some embodiments, the angle of the extraction tip is adjustable using a cable, rod, or other wiring mechanism. In this manner, a healthcare practitioner is able to position the extraction tip behind cerumen or other objects where there is insufficient clearance to move the full height of the extraction tip behind the cerumen or other object. In some embodiments, the extraction tip is a set of forceps that open and close to grip an object according to movement (e.g., pulling or pushing) of the cable, rod, or other wiring mechanism.

### BRIEF DESCRIPTION OF THE DRAWINGS

In order to achieve a better understanding of the nature of the present invention a preferred embodiment of the motorized cutting tool attachment will now be described, by way of example only, with reference to the accompanying drawings in which:

FIG. 1 illustrates an otoscope with a removable cover.

FIG. 2 illustrates an attachment that is an enhanced cover for an otoscope to facilitate examination and object extraction using the otoscope in accordance with some embodiments of the invention.

FIG. 3 illustrates the attachment coupling to the attachment assembly of the otoscope in accordance with some embodiments.

FIG. 4 illustrates the support extending at an angle away from the focal tip in accordance with some embodiments.

FIG. 5 illustrates the support extending at an angle towards the focal tip in accordance with some embodiments.

FIG. 6 illustrates a retractable support for the attachment in accordance with some embodiments.

FIG. 7 illustrates the extraction tip as a circular or oblong shaped solid scoop in accordance with some embodiments.

FIG. 8 illustrates the extraction tip as a square or rectangular shaped solid scoop in accordance with some embodiments.

FIG. 9 illustrates the extraction tip as a set of bristles in accordance with some embodiments.

FIG. 10 illustrates various angles at which the extraction tip may be located at the end of the support in accordance with some embodiments.

FIGS. 11-13 illustrate using the attachment to visualize a small orifice and remove an object from the orifice in accordance with some embodiments.

FIG. 14 illustrates an attachment with a moving extraction tip in accordance with some embodiments.

FIG. 15 movement of the moving extraction tip in accordance with some embodiments.

FIG. 16 illustrates a forceps extraction tip for the attachment in accordance with some embodiments.

FIG. 17 illustrates use of the forceps extraction tip in accordance with some embodiments.

### DETAILED DESCRIPTION OF THE INVENTION

In the following detailed description of the invention, numerous details, examples, and embodiments of the invention are set forth and described. However, it will be clear and apparent to one skilled in the art that the invention is not limited to the embodiments set forth and that the invention may be practiced without some of the specific details and examples discussed.

Some embodiments provide an attachment for adapting an examination tool to identify an object within an orifice or other opening and to safely extract the object using the examination tool. In some embodiments, the attachment is an enhanced otoscope cover that facilitates object extraction using an otoscope without the need for a curette or other specialized tools.

An otoscope is an examination tool used by many healthcare practitioners to examine small orifices and other bodily openings. The otoscope is a probe that lights and magnifies a view of the orifice or opening. The otoscope is commonly used to examine the ear canal and to identify blockages within the ear canal. Blockages in the ear canal may be the result of excess cerumen or excess ear wax buildup. Other blockages may be caused by foreign objects (e.g., insects, dirt, etc.) entering the ear canal.

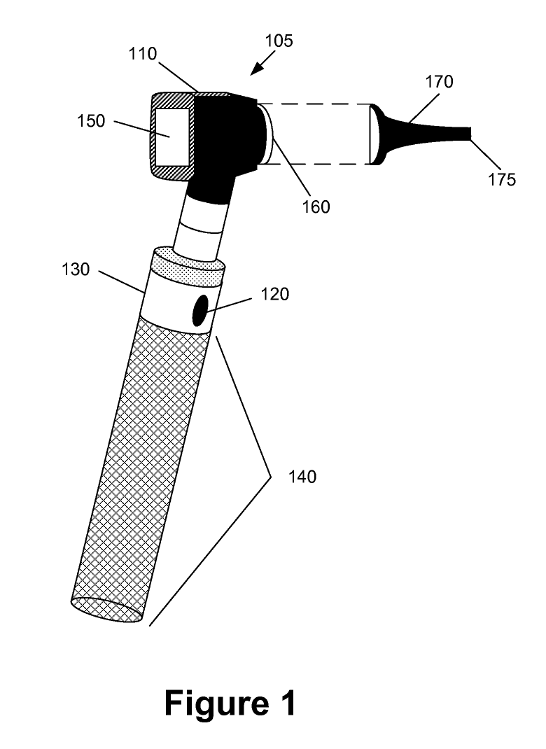

FIG. 1 illustrates an otoscope 105 with a removable cover 170. The otoscope 105 includes head 110, trigger 120, rotating member 130, and body 140. The head 110 includes viewing window 150, attachment assembly 160, and a light. The light is contained within the head 110 and is therefore not shown in FIG. 1.

The attachment assembly 160 allows for different covers to couple to the otoscope head 110. In this figure, cover 170 is shown coupling to the attachment assembly 160. The attachment assembly 160 may include grooves that align with grooves within the cover 170 to hold the cover 170 stationary. Other attachment assemblies 160 may include a screw assembly or a push button assembly for coupling the cover to the otoscope head 110. Accordingly, it should be apparent that the attachment assembly 160 may include other means for coupling the cover to the otoscope head 110.

Otoscope covers (e.g., cover 170) are typically conically shaped or funnel shaped so as to provide a focal tip (e.g., focal tip 175) that has a smaller circumference than the base of the cover. This allows the focal tip of the cover to be inserted into small orifices such as the ear canal. In this figure, cover 170 with focal tip 175 is shown attaching to the attachment assembly 160. In some instances, the attachment assembly 160 includes a default focal tip providing a default view at the viewing window 150 which can be adjusted by coupling differently shaped covers to the attachment assembly 160.

The size and shape of the focal tip determines the view at the viewing window 150. Specifically, the viewing window 150 provides a view of what can be seen through the focal tip. Covers of different shapes and lengths may be used to alter the focal tip and thus alter the view from the viewing window 150. A focal tip with a smaller circumference provides a more restricted view than a focal tip with a larger circumference. However, the focal tip with the smaller circumference can be inserted into smaller orifices or openings than the focal tip with the larger circumference. Similarly, covers that are longer in length can be inserted deeper into the orifice or opening.

In some instances, the viewing window 150 includes a magnification lens to magnify the view at the focal tip. Depending on the otoscope manufacturer, the amount of magnification may be adjustable at the head 110.

Trigger 120 turns on and off the light within the head 110 which is used to illuminate the view from the focal tip. The focal tip focuses the light from the otoscope head 110 into the orifice or opening in which the cover is inserted. In some instances, the rotating member 130 adjusts the intensity or brightness of the light. The body 140 of the otoscope 105 is a cylindrical shaft that houses the batteries that power the light.

The viewing window 150 therefore provides a magnified and illuminated view of the orifice or opening. In this manner, the healthcare practitioner is able to examine the orifice or opening in order to identify blockages or objects for removal. Welch Allyn® is a manufacturer of otoscopes similar to otoscope 105.

Some embodiments provide an attachment to enhance the otoscope functionality by adapting the otoscope to simultaneously perform examination and object extraction without the need for curettes or other specialized tools. The attachment is an enhanced cover with an extraction tip that couples to the otoscope. The attachment leverages the magnification and lighting functionalities of the otoscope to examine the ear canal and other small orifices and identify blockages therein as is done with traditional covers. The attachment includes the extraction tip whereby any identified blockages may be cleared using the attachment that is already coupled to the otoscope without modification to the otoscope, modification to the attachment, or use of curettes or other specialized tools.

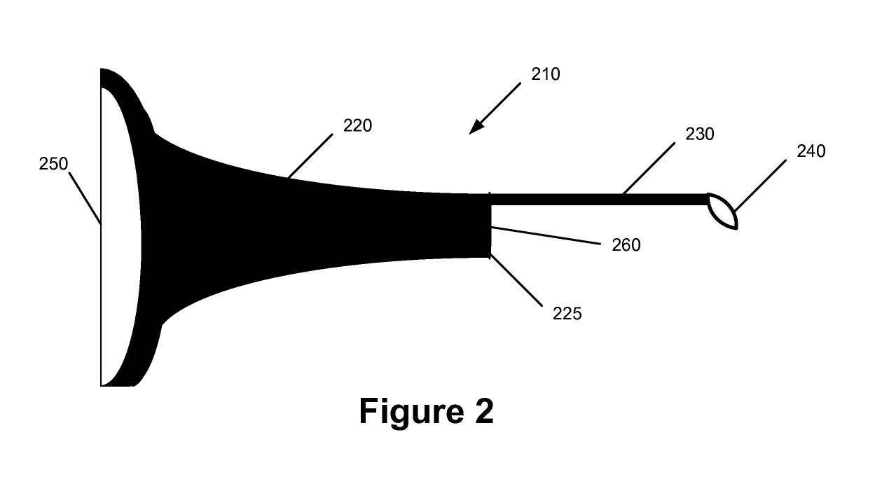

FIG. 2 illustrates an attachment 210 that is an enhanced cover for an otoscope to facilitate examination and object extraction using the otoscope in accordance with some embodiments of the invention. The attachment 210 includes cover 220, support 230, and extraction tip 240.

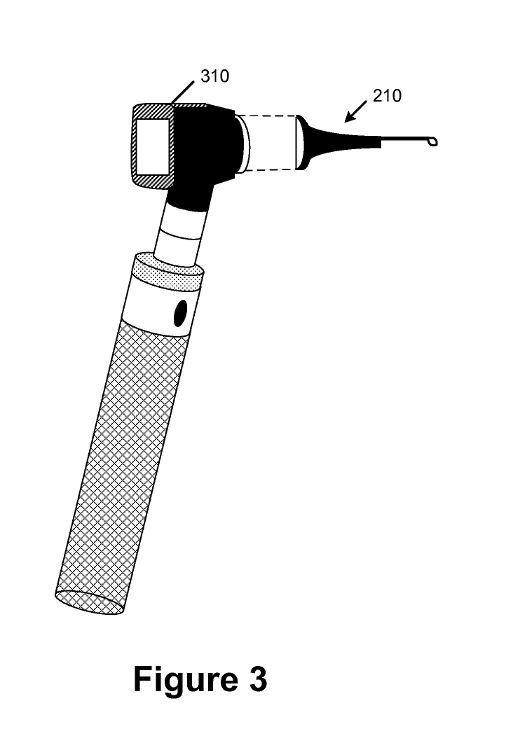

The cover 220 couples the attachment 210 to the attachment assembly of the otoscope. The cover 220 is a hood in the form of a tubular membrane with two openings, the first opening 250 being wider in circumference than the second opening 260. In some embodiments, the cover 220 contains grooves near the first opening 250 that align with grooves on the attachment assembly of the otoscope. These grooves serve to secure the attachment 210 to the head of the otoscope. FIG. 3 illustrates the attachment 210 coupling to the attachment assembly of the otoscope 310 in accordance with some embodiments. It should be apparent that the cover 220 may include other means for coupling the attachment 210 to the attachment assembly of the otoscope. Depending on the otoscope design or manufacturer, the cover may include a screw base or push button assembly for coupling the attachment 210 to the attachment assembly of the otoscope.

In some embodiments, the tubular membrane of the cover 220 is conically shaped to provide a focal tip 225 at the second opening 260. The shape gradually reduces the circumference of the cover 220 from the first opening 250 to the focal tip 225 at the second opening 260. The circumference at the focal tip 225 is small enough so that a portion of the cover 220 may be inserted into small orifices such as the ear canal. Additionally, the shape of the cover 220 focuses light from the first opening 250 to the second opening 260. In this manner, the light from the otoscope head shines through the focal tip 225 and illuminates the volume of the small orifice viewable from the focal tip 225. In some embodiments, the circumference of the cover 220 ranges from 5-30 millimeters (mm) with the focal tip 225 having a smaller circumference than the first opening 250. In some embodiments, the cover 220 is up to 60 mm in length. It should be apparent to one of ordinary skill that the circumference of the cover 220 may remain the same from the first opening 250 to the focal tip 225 at the second opening 260.

In some embodiments, the support 230 extends 1-50 mm from the focal tip 225 of the cover 220. The support 230 may be located at any position around the focal tip 225 (e.g., top, right, left, etc.). In some embodiments, the support 230 extends at an angle away from the focal tip 225, at an angle towards the focal tip 225, or laterally from the focal tip 225 with no angle.

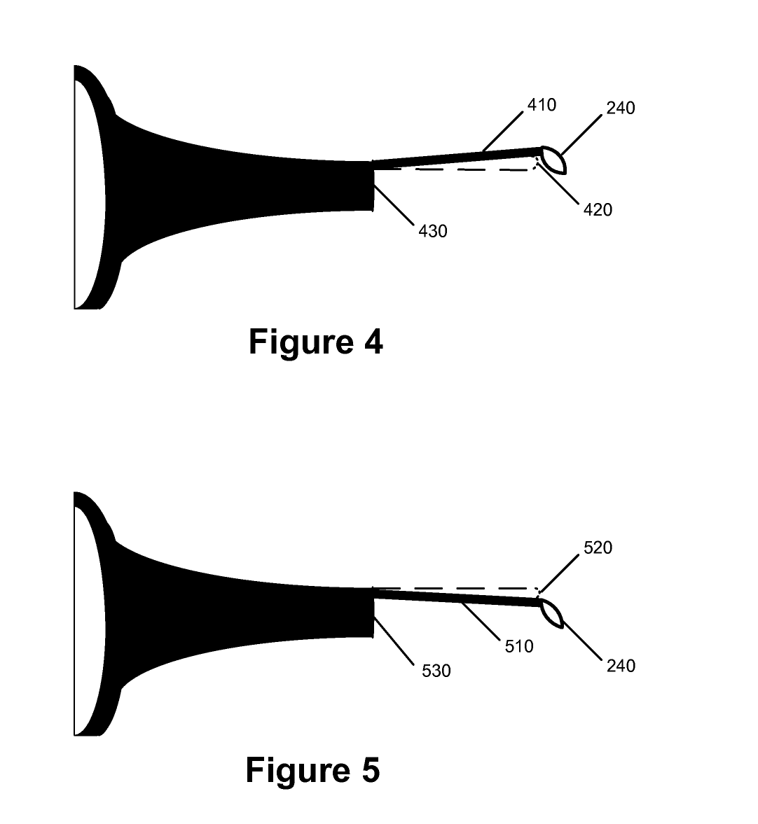

FIG. 4 illustrates the support 410 extending at an angle 420 away from the focal tip 430 in accordance with some embodiments. In some embodiments, the incline of the angle 420 away from the focal tip 430 is between 1 and 45 degrees. By angling the support away from the focal tip 430, the extraction tip 240 is less visible through the focal tip 430 and from the viewing window of the otoscope.

FIG. 5 illustrates the support 510 extending at an angle 520 towards the focal tip 530 in accordance with some embodiments. In some embodiments, the angle 520 of the support is between 1 and 45 degrees towards the focal tip 530. By angling the support into the focal tip 530, the extraction tip 240 is more visible through the focal tip 530 and from the viewing window of the otoscope.

The different angles of the support (e.g., supports 230, 410, and 510) position the extraction tip differently, thereby providing different views of the extraction tip through the viewing window of the otoscope. The different angles of the support (e.g., supports 230, 410, and 510) allow a healthcare practitioner to use the attachment in a manner that is customized to his/her comfort.

In some embodiments, the support (e.g., supports 230, 410, and 510) is rigid so to prevent bending upon contact with a solid object (e.g., a wall of the ear canal). In some other embodiments, the support (e.g., supports 230, 410, and 510) are flexible so that upon contact with a solid object, the support flexes by some degree (e.g., 1 to 15 degrees) from a stationary position. Some embodiments provide different degrees of rigidity and flex. Flex provides greater maneuverability of the extraction tip in cramp spaces. Rigidity provides greater precision for the extraction tip as the support and thus by extension the extraction tip do not bend or move in cramp spaces.

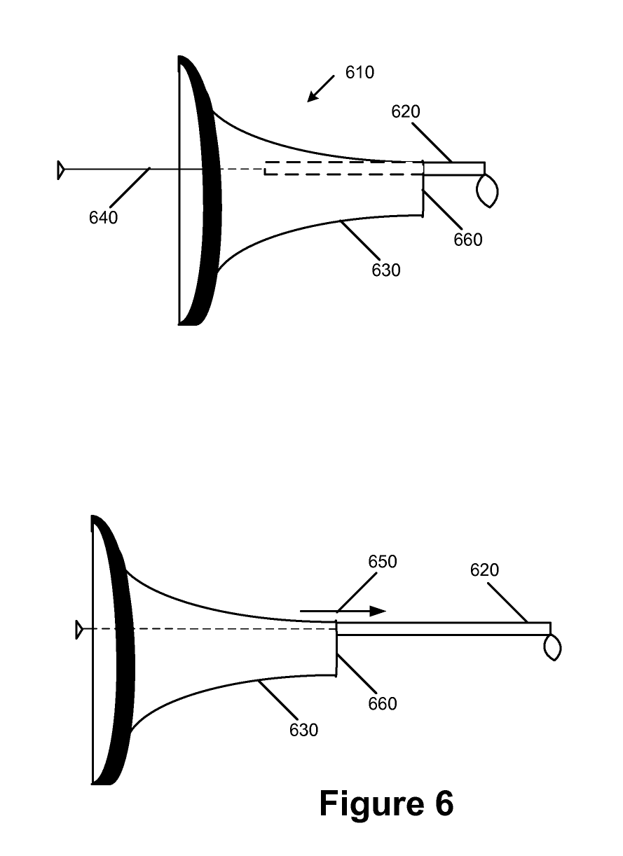

In some embodiments, the support is retractable. FIG. 6 illustrates a retractable support 620 for the attachment 610 in accordance with some embodiments. The support 620 slides along either the inside or outside of the cover 630 using retraction mechanism 640. In FIG. 6, the support 620 is shown sliding into the cover 630. In some embodiments, the retraction mechanism 640 is made of a rigid wire or rod that attaches to the support 620. The wire or rod may be composed of thread, plastic, metal, or other materials. The retraction mechanism 640 is manually operated by a healthcare practitioner.

When the retraction mechanism 640 is pushed a distance 650 away from the base of the cover 630 towards the focal tip 660, the support 620 slides out from the cover 630 and extends outward from the focal tip 660. When the retraction mechanism 640 is pulled inwards from the focal tip 660 towards the base of the cover 630, the support 620 slides along the inside of the cover 630 and retracts into the cover 630. In this manner, the support and extraction tip may be moved so as to not hinder probing of an orifice using the attachment when the attachment is used to examine and visualize. In some embodiments, the retraction mechanism 640 includes means for fixing the position of the support 620 at any point along the cover 630.

With reference back to FIG. 2, the extraction tip 240 is located at the end of the support 230 and is angled to be visible through the focal tip 225. In this configuration, the healthcare practitioner can manipulate the otoscope and thereby manipulate the position of the extraction tip 240 in order to engage cerumen and other objects from behind. Once engaged, the extraction tip is pulled thereby creating a traction force which extracts the cerumen or other objects.

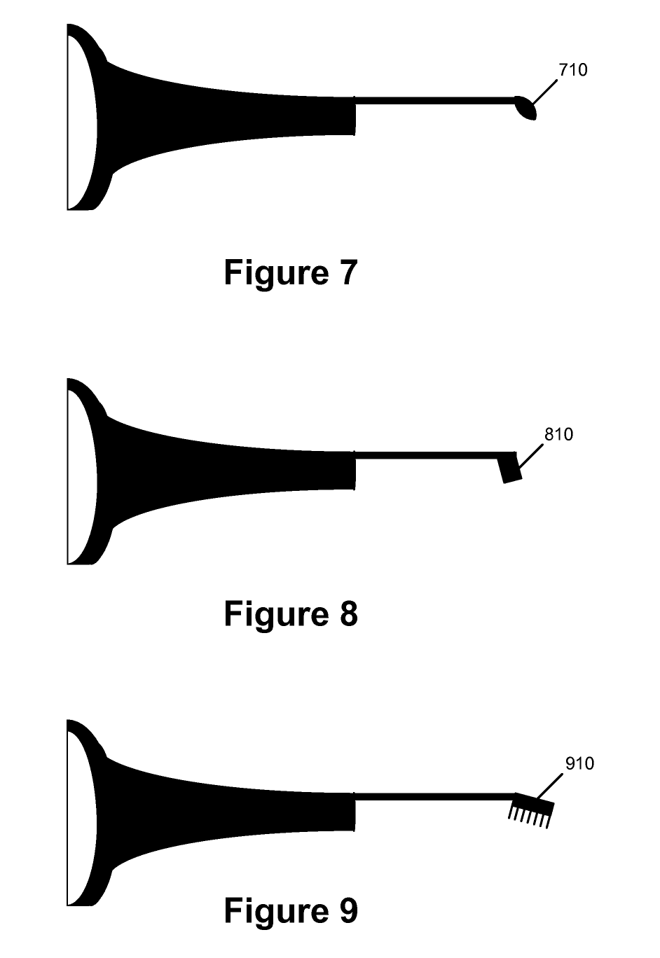

The extraction tip may be of various shapes. In FIG. 2, the extraction tip 240 is a circular or oblong hollow scoop. In this configuration, light from the otoscope is able to pass through the cover 220 and the extraction tip 240. FIGS. 7-9 illustrates various other shapes of the extraction tip in accordance with some embodiments.

FIG. 7 illustrates the extraction tip 710 as a circular or oblong shaped solid scoop in accordance with some embodiments. FIG. 8 illustrates the extraction tip 810 as a square or rectangular shaped solid scoop in accordance with some embodiments. FIG. 9 illustrates the extraction tip 910 as a set of bristles in accordance with some embodiments. FIGS. 7-9 illustrates some examples of the extraction tip that may be used in conjunction with the attachment of some embodiments. However, it should be apparent that additional shapes or different combinations of the shapes may be used for the extraction tip and that FIGS. 7-9 provided some such examples. For instance, the extraction tip may include a square or rectangular shaped hollow scoop.

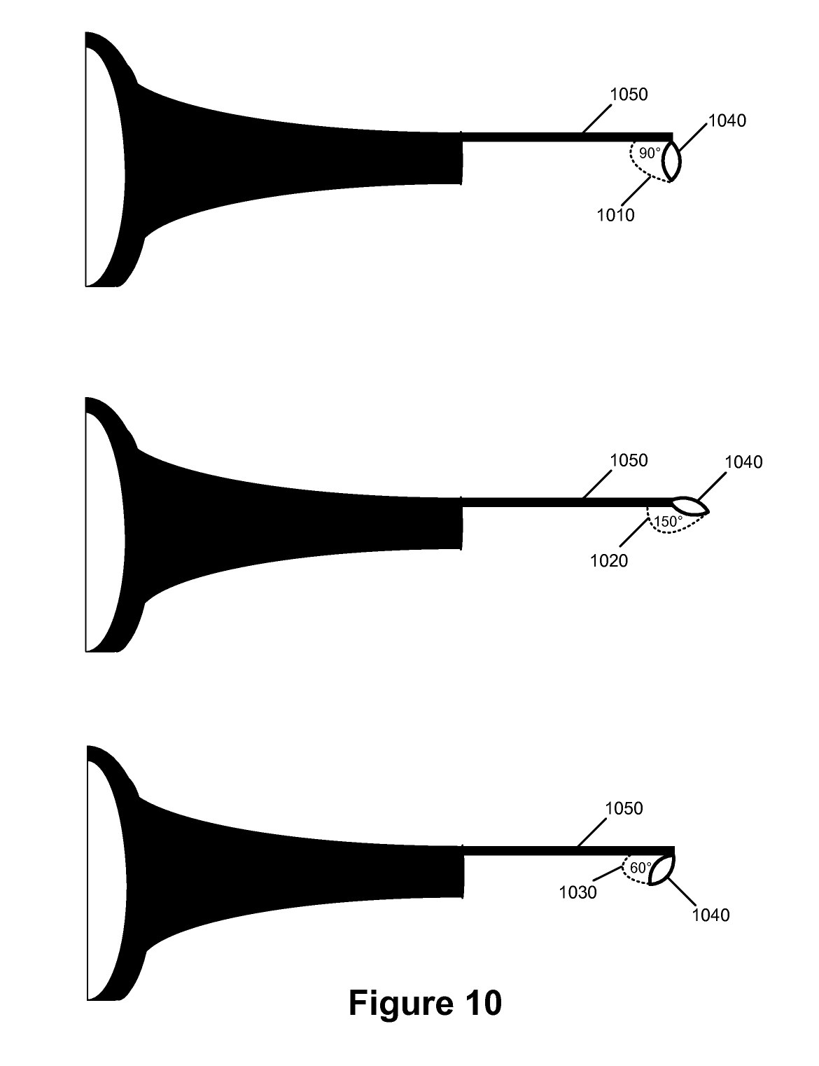

In some embodiments, the extraction tip 240 is 2-20 mm in width, 2-20 mm in height, and 1-4 mm in thickness. FIG. 10 illustrates various angles 1010-1030 at which the extraction tip 1040 may be located at the end of the support 1050 in accordance with some embodiments. For example, angle 1010 illustrates the extraction tip 1040 angled 90 degrees from the support 1050, angle 1020 illustrates the extraction tip 1040 angled 150 degrees from the support 1050, and angle 1030 illustrates the extraction tip 1040 angled 60 degrees from the support 1050. In some embodiments, the angle of the extraction tip 1040 varies from 10 to 170 degrees from the position of the support 1050 where a 90 degree angle of the extraction tip 1040 positions the extraction tip 1040 perpendicular to the support 1050.

In some embodiments, the extraction tip (e.g., extraction tip 240 and 1040) provides some degree of flex when contacting a rigid object. This allows the extraction tip to bend if needed providing greater mobility for the extraction tip. For example, the extraction tip may bend to fit the curvature of the ear canal without damaging the ear canal. In some embodiments, the extraction tip flexes 1-15 degrees from its stationary position. In some other embodiments, the extraction tip is rigid so to prevent bending upon contact with an object (e.g., cerumen). A rigid extraction tip provides greater traction force when removing an object as the force used to manipulate the otoscope is directly translated to the rigid extraction tip.

The attachment of some embodiments provides several advantages over traditional speculum covers, curettes, and other specialized tools. With the attachment 210 coupled to an otoscope, the healthcare practitioner can examine the ear canal for blockages in the same manner as with a traditional speculum cover coupled to the otoscope. However unlike traditional speculum covers, the healthcare practitioner can manipulate the position of the attachment 210 using the otoscope in order to move the extraction tip 240 behind any identified objects (e.g., cerumen, dirt, etc.) that are causing a blockage. These objects can be removed by pulling the otoscope out from the ear, causing the blockage to come out with the extraction tip 240. In this manner, the healthcare practitioner is able to remove objects from small orifices in a manner previously not possible with existing otoscope covers and without the need for curettes and other specialized tools. The attachment reduces medical cost since less time is needed to perform the extraction. The healthcare practitioner can visualize and remove cerumen and other objects from small orifices using the primary examination tool, the otoscope.

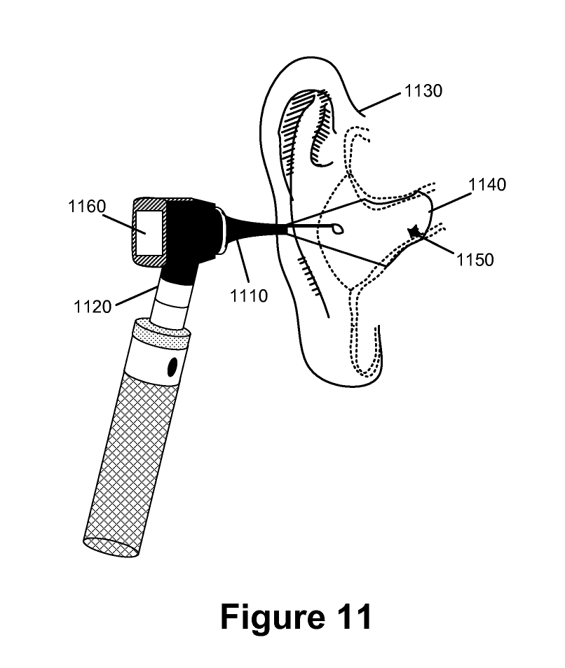

FIGS. 11-13 illustrate using the attachment 1110 to visualize a small orifice and remove an object from the orifice in accordance with some embodiments. The visualization and removal are shown as three separate figures (FIGS. 11, 12, and 13) with each figure representing a different stage of the visualization and removal.

In FIG. 11, the attachment 1110 is coupled to an otoscope 1120 and the attachment 1110 is inserted into the ear 1130 in order to examine the ear 1130, ear canal, or ear drum. The otoscope 1120 contains a light that shines through the attachment 1110 and the focal tip of the attachment 1110. The light illuminates volume 1140 in and around the ear 1130. Volume 1140 is viewable from the focal tip of the attachment 1110. Accordingly, the viewing window 1160 of the otoscope 1120 provides a magnified view of the illuminated volume 1140. From the viewing window 1160, a healthcare practitioner identifies object 1150 as causing a blockage within the ear 1130.

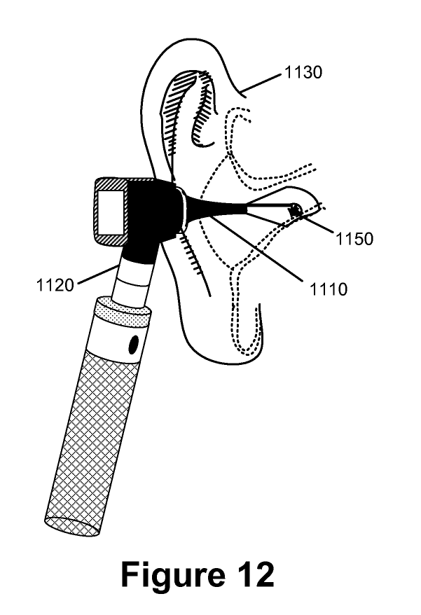

In FIG. 12, the healthcare practitioner manipulates the position of the attachment 1110 in order to remove the object 1150. Specifically, the healthcare practitioner positions the extraction tip of the attachment 1110 behind the object 1150.

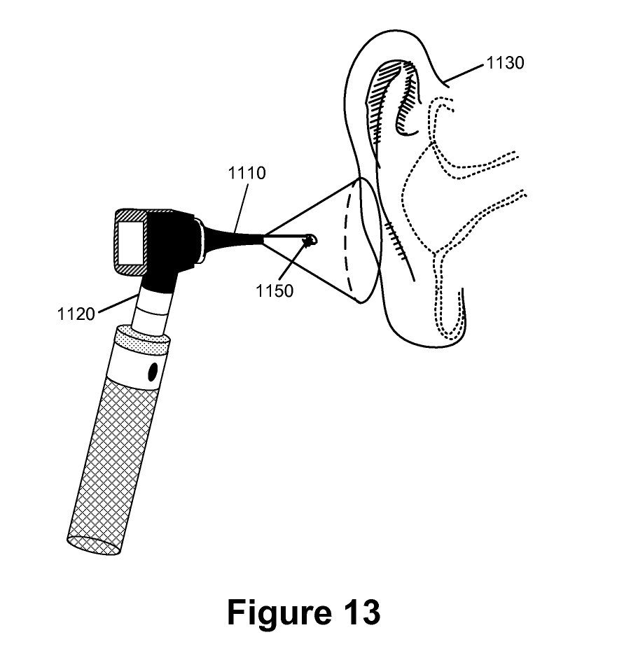

In FIG. 13, the otoscope 1120 is removed from the ear 1130. As the otoscope 1120 is pulled outwards, the extraction tip of the attachment 1110 engages the object 1150 and pulls the object 1150 out from the ear 1130.

The attachment 1110 may be cleaned and sanitized for reuse. Alternatively, the attachment 1110 may be detached, discarded, and replaced with a new attachment or other speculum cover.

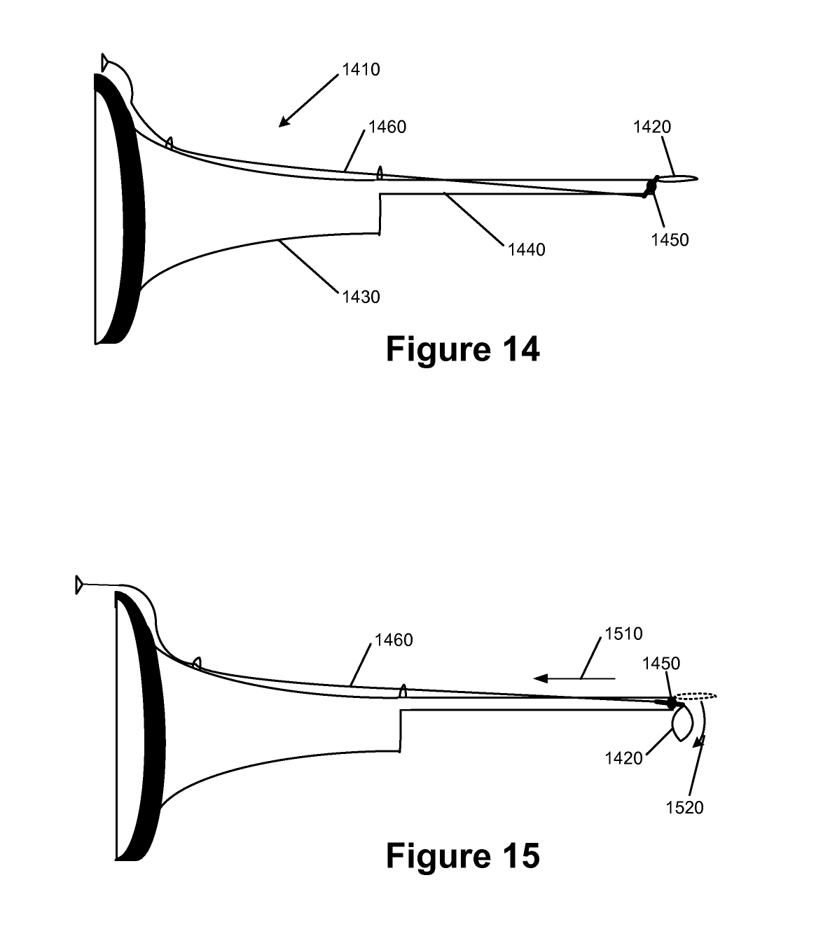

To assist in positioning the extraction tip of the attachment behind an object that is to be removed, some embodiments provide a moving extraction tip. The movement of the extraction tip is controlled by the healthcare practitioner using a thin rod or wiring mechanism that runs along the body of the attachment. FIGS. 14 and 15 illustrate an attachment 1410 with a moving extraction tip 1420 in accordance with some embodiments.

As before, the attachment 1410 of FIG. 14 includes cover 1430, support 1440, and extraction tip 1420. The attachment 1410 also includes pivot point 1450 and wiring mechanism 1460. The pivot point 1450 is a rotating element located along the support 1440. In some embodiments, the pivot point 1450 is coupled to the extraction tip 1420 at one end and to the wiring mechanism 1460 at an opposite end.

FIG. 14 illustrates the extraction tip 1420 at a default position away from the focal tip of the cover 1420. However in FIG. 15, pulling the wiring mechanism 1460 by a distance 1510 causes the pivot point 1450 to rotate. This rotation also causes the extraction tip 1420, which is coupled to the pivot point 1450, to rotate by a distance 1520. The rotation 1520 of the extraction tip 1420 positions the extraction tip 1420 closer towards the focal tip and therefore more within view from the focal tip. In this position, the healthcare practitioner uses the extraction tip 1420 to engage and remove objects from behind.

In some embodiments, the healthcare practitioner manually returns the extraction tip 1420 to the default position (i.e., the position of the extraction tip 1420 in FIG. 14). The healthcare practitioner grabs the extraction tip 1420 and rotates it away from the focal tip.

In some embodiments, the wiring mechanism 1460 is a rigid rod (e.g., metal or plastic) that can be pushed away from the base of the cover 1430 (i.e., opposite to the direction 1510) in order to reposition the extraction tip 1420 at its default position away from the focal tip (i.e., the position as shown in FIG. 14). Specifically, pushing the wiring mechanism 1460 causes the pivot point 1450 to rotate in the opposite direction to that of 1520. The rotation of the pivot point 1450 moves the extraction tip 1420 away from the focal tip.

In some embodiments, the wiring mechanism 1460 includes a spring that is coupled to the pivot point 1450 and wiring. The spring provides a default position for the extraction tip 1420 when there is no tension on the wiring. Pulling the wiring creates tension on the spring which causes the pivot point 1450 to rotate and the extraction tip 1420 to move. Once the wiring is released, the spring automatically causes the pivot point 1450 to rotate in the opposite direction, bringing the extraction tip 1420 back to its default position. In some embodiments, the default position of the extraction tip 1420 is away from the focal tip as shown in FIG. 14.

Using the movable extraction tip of some embodiments, the healthcare practitioner is better able to position the extraction tip 1420 behind cerumen. For example, to position a fixed or rigid extraction tip behind an object within a space, there must be sufficient clearance within the space for the height of the extraction tip to fit around the object. However, with the moving extraction tip, the extraction tip may be positioned so that less clearance is needed within the space in order to position the extraction tip behind the object. In FIG. 14, the extraction tip 1420 is positioned in a manner whereby clearance equal to or greater than the thickness of the extraction tip 1420 is sufficient to move the extraction tip 1420 behind an object. Once behind the object, the extraction tip 1420 can be rotated downwards as in FIG. 15 so as to engage and remove the object.

FIGS. 14 and 15 illustrate the moving extraction tip as a hollow circular/oblong scoop. It should be apparent to one of ordinary skill in the art that the moving extraction tip may be of various shapes and sizes including but not limited to those depicted in FIGS. 7-9. It should be apparent to one of ordinary skill in the art that a motorized mechanism at the end of the support may be used to automatically adjust the position of the extraction tip.

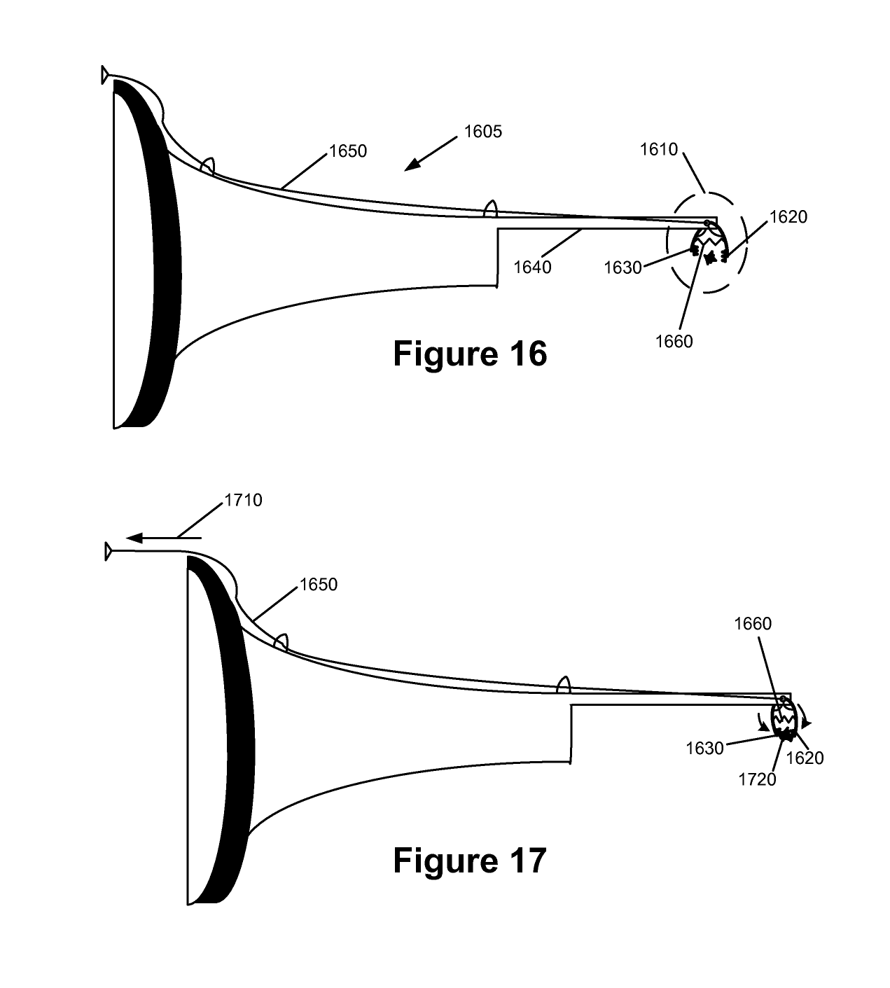

In some embodiments, the extraction tip provides different means of extraction. FIG. 16 illustrates a forceps extraction tip 1610 for the attachment 1605 in accordance with some embodiments. The forceps extraction tip 1610 includes a first prong 1620 extending over a first side of the support 1640, a second prong 1630 extending over a second side of the support 1640 that is opposite to the first side, and wiring mechanism 1650. In some embodiments, the first side of the support 1640 is the right side of the support 1640 and the second side of the support 1640 is the left side of the support 1640. The prongs 1620 and 1630 include pads or other grasping surface to prevent slippage when gripping an object.

The wiring mechanism 1650 is coupled to the first and second prongs 1620 and 1630 and runs along the support 1640 and the attachment 1605. The wiring mechanism 1650 controls the opening and closing of the prongs 1620 and 1630 as is shown in further detail below with reference to FIG. 17. In some embodiments, the wiring mechanism 1650 includes a wire, rod, or other rigid control for controlling the opening and closing of the prongs 1620 and 1630.

In some embodiments, the forceps extraction tip 1610 further includes a spring 1660 that retains the prongs 1620 and 1630 in a default open position when there is no tension on the wiring mechanism 1650 and allows the prongs 1620 and 1630 to close around an object when there is tension on the wiring mechanism 1650.

As shown in FIG. 17, pulling the wiring mechanism 1650 a distance 1710 causes the prongs 1620 and 1630 to close around object 1720. Specifically, the wiring mechanism 1650 includes a wire that couples to the inside of each of the prongs 1620 and 1630. When pulled, the wire becomes taught and the force of the wire moves the prongs 1620 and 1630 towards each other thereby griping the object 1720. The object 1720 may then be extracted by retaining the prongs 1620 and 1630 in a closed position and by pulling the attachment or medical examination tool to which the attachment is coupled outwards.

In the closed position, the spring 1660 is compressed. Therefore, once the tension on the wire is released, the spring will decompress and return the prongs to the open position. In some embodiments, the spring 1660 retains the prongs 1620 and 1630 in a default closed position when there is no tension on the wiring mechanism 1650 and allows the prongs 1620 and 1630 to open when there is tension.

The forceps extraction tip 1610 provides an alternative means for removing cerumen and other objects from various orifices. Specifically, the forceps extraction tip 1610 engages an object from the front. In this manner, object extraction occurs by gripping the object and pulling the object as opposed to pushing the object from behind.

The attachment has been described as an enhanced otoscope cover for examining and extracting objects (e.g., cerumen) from orifices such as the ear canal. It should be apparent to one of ordinary skill in the art that the attachment adapts the otoscope for examination and extraction of objects from any openings where the light and magnification functionalities of the otoscope assist in object extraction. For example, healthcare practitioners may utilize the attachment to remove fragments (e.g., dead tissue, dirt, mucus, etc.) from open body wounds, to obtain biopsy samples from incisions made to the body, to examine and remove blockages from the nose and nasal cavity, and to scrape or dislodge objects from other such openings. For some such procedures, the dimensions of the attachment cover, support, or extraction tip may be smaller or larger than those described herein.

Accordingly, the attachment is not limited to the extraction of blockages from the ear canal or for use within the ear canal and may be used generally in the course of the healthcare practitioner's duties. Moreover, the attachment may be adapted for non-medical uses. For example, gemologists may utilize the attachment to examine gems and remove dirt or other objects from the gems using the extraction tip.

In some embodiments, the attachment is made from a rigid material such as metal, plastic, or other polymer. When composed of plastic, the attachment is inexpensive to manufacture relative to a curette and may be either disposable or reusable. The enhanced otoscope cover of some embodiments will allow significant improvement in the ease with which healthcare practitioners remove wax out of the ears of patients. The enhanced otoscope cover of some embodiments will reduce medical costs by eliminating the need for multiple tools to remove wax as the same enhanced speculum cover can be used to visualize the ear and remove wax if it is found.

While the invention has been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

1. An attachment for an examination tool, said attachment comprising:
a cover comprising:
(i) a base for coupling said attachment to the examination tool,
(ii) a tubular membrane for insertion into an opening, and
(iii) a focal tip for viewing into said opening;
a support extending a specified distance from said cover; and
a scoop extending at an angle from the support into said view from the focal tip, said scoop for removing an object.

2. The attachment of claim 1, wherein said scoop removes said object by contacting said small object from behind and pulling said object out from said opening.

3. The attachment of claim 1, wherein said scoop comprises at least one of: a circular hollow scoop, a rectangular hollow scoop, a circular solid scoop, and a rectangular solid scoop.

4. The attachment of claim 1, wherein said scoop comprises a set of bristles for brushing said object.

5. The attachment of claim 1, wherein said scoop comprises a set of forceps for grasping said object using a set of moveable arms.

6. The attachment of claim 1 further comprising a wiring mechanism for adjusting the angle at which the scoop extends from the support.

7. The attachment of claim 6, wherein said wiring mechanism adjusts the angle of the scoop from 10 to 170 degrees from a position of the support.

8. The attachment of claim 6, wherein moving said wiring mechanism in a first direction decreases the angle of the scoop relative to the support and moving said wiring mechanism in a second direction increases the angle of the scoop relative to the support.

9. The attachment of claim 1, wherein said examination tool comprises an otoscope.

10. The attachment of claim 1, wherein said scoop flexes 1 to 15 degrees upon contacting a rigid object.

11. The attachment of claim 1, wherein said support flexes 1 to 15 degrees upon contacting a rigid object.

12. The attachment of claim 1 further comprising a retraction mechanism for retracting said support into the cover and for extending said support out from the cover.

13. An attachment for performing object removal using an otoscope, said attachment comprising:
a tubular hood comprising first and second openings, wherein the first opening is adapted to couple said attachment to the otoscope; and
an extraction tip extending a distance from the second opening of the hood, said extraction tip for engaging and removing an object through manipulations of the otoscope.

14. The attachment of claim 13, wherein said extraction tip comprises at least one of: a circular hollow scoop, a rectangular hollow scoop, a circular solid scoop, a rectangular solid scoop, a set of bristles, and a set of forceps.

15. The attachment of claim 13, wherein the first opening of the tubular hood is wider than the second opening to allow for the second opening to be inserted into a small opening.

16. A method for performing cerumen extraction using an otoscope and an enhanced otoscope cover with an extraction tip, the method comprising:
inserting said enhanced otoscope cover into an opening;
identifying an object within said opening for removal using a viewing window of the otoscope that provides a view through the enhanced otoscope cover;
positioning said extraction tip behind said object through manipulations of the otoscope; and
extracting said object by maintaining the position of the extraction tip behind said object and by removing the otoscope from said opening.

17. The method of claim 16, wherein positioning said extraction tip comprises adjusting an angle of the extraction tip.

18. The method of claim 16, wherein identifying the object comprises using magnification and light from the otoscope to enhance the view through the enhanced otoscope cover.
